(19) United States
(12) Patent Application Publication
(10) Pub. No.: US 2025/0364146 A1
(43) Pub. Date: Nov. 27, 2025

(54) REALTIME MODELING OF ANATOMICAL PHYSICS

(71) Applicants: Boston Scientific Scimed, Inc., Maple Grove, MN (US); Boston Scientific Medical Device Limited, Galway (IE)

(72) Inventors: Jessica WILLIAMS, Marlborough, MA (US); Longquan CHEN, Lexington, MA (US); Arun ADHIKARATH BALAN, Aluva (IN); Niraj Prasad RAUNIYAR, Plymouth, MN (US); Jeffrey A. MEGANCK, North Grafton, MA (US); Elizabeth A. STOKLEY, Raleigh, NC (US); Mihir SUKHATME, Thane (IN); Samriddhi JAIN, Delhi (IN)

(73) Assignees: Boston Scientific Scimed, Inc., Maple Grove, MN (US); Boston Scientific Medical Device Limited, Galway (IE)

(21) Appl. No.: 19/215,726

(22) Filed: May 22, 2025

Related U.S. Application Data
(60) Provisional application No. 63/651,523, filed on May 24, 2024.

(51) Int. Cl. G16H 50/50; G06F 3/0482

(52) U.S. Cl. CPC G16H 50/50; G06F 3/0482

(57) ABSTRACT

Medical systems are described, including a medical system including a display having a user interface that includes graphical elements corresponding to a first set of medical parameters and a type of intraluminal state, and a processor configured to receive user input selecting a status or value for at least one medical parameter of the first set of medical parameters and the type of intraluminal state, determine the intraluminal state over time based on the user input, and display on the user interface a graphical representation of the intraluminal state over time.

### CROSS-REFERENCE TO RELATED APPLICATION(S)

This patent application claims the benefit of priority to U.S. Provisional Application No. 63/651,523, filed on May 24, 2024, which is incorporated by reference herein in its entirety.

### TECHNICAL FIELD

The disclosure relates generally to devices, systems, and methods for medical procedures. More specifically, aspects of the disclosure pertain to devices, systems, and/or methods for visualization and analysis of anatomical physics involved in medical procedures such as intraluminal pressures during irrigation.

### BACKGROUND

Medical procedures that use irrigation to improve visualization of patient anatomy may involve pressurization of a working space (e.g., at a target site in the patient). Irrigation of a target site may be performed by an operator to clear a field of view for enhanced visual clarity via an imaging device such as a camera. Introduction of fluid to a target site may lead to increased intraluminal pressure (ILP). Increased ILP may be associated with adverse patient outcomes. For example, pyelovenous backflow in the renal pelvis may be caused by high pressure (e.g., caused by irrigation) and may also result in sepsis. Additionally, procedural factors such as using lasers internally, may result in increased temperatures which can cause damage to surrounding tissue of a patient.

Measuring ILP, flowrate, and/or temperature during medical procedures presents challenges. Further, medical professionals may be unfamiliar how several physical parameters may impact ILP, flowrate, and/or temperature over the course of a procedure.

### SUMMARY

Examples of the present disclosure relate to, among other things, systems, devices, and methods for visualization and analysis of anatomical physics involved in medical procedures such as intraluminal pressures during irrigation. Each of the examples disclosed herein may include one or more of the features described in connection with the disclosed examples.

For example, the present disclosure includes a medical system having a display including a user interface that includes graphical elements corresponding to a first set of medical parameters and a type of intraluminal state, and a processor configured to receive user input selecting a status or value for at least one medical parameter of the first set of medical parameters and the type of intraluminal state, determine the intraluminal state over time based on the user input, and display on the user interface a graphical representation of the intraluminal state over time.

The type of intraluminal state may be intraluminal pressure, fluid flow rate, or body temperature, e.g., renal temperature or the temperature of another organ or other portion of the body undergoing a medical procedure). The first set of medical parameters may include at least two medical parameters chosen from a kidney compliance, a ureter size, an access sheath size, a tool designation, a fluid temperature, a laser setting, a fluid bag height, or a cuff pressure. In some examples, the first set of medical parameters may include a ureter size, an access sheath size, a fluid temperature, and/or a laser setting, and the type of intraluminal state may be intraluminal pressure or body temperature, e.g., renal temperature. The user interface may include a default value or status for each medical parameter of the first set of medical parameters, the default value or status capable of being modified by a user. According to some aspects, the medical system may further include a memory that stores the default value or status for each medical parameter. Determining the intraluminal state over time may include, e.g., numerically solving differential equations based on the user input selecting the status or value for the at least one medical parameter. The processor may be further configured to receive user input selecting static visualization or dynamic visualization of the intraluminal state and/or to receive user input selecting dynamic visualization, and to update the graphical representation based on user input changing a value or status of at least one medical parameter. The user interface further may include a graphical element corresponding to a fluid flush, and the user input may include a selection of the fluid flush. The user interface may include graphical elements corresponding to a second set of medical parameters that are the same as the medical parameters of the first set of medical parameters. The intraluminal state may be a first intraluminal state and the processor may be further configured to receive user input selecting a status or value for at least one medical parameter of the second set of medical parameters, determine a second intraluminal state over time based on the user input of the status or value for the at least one medical parameter of the second set of medical parameters, and update the user interface to overlay a graphical representation of the second intraluminal state over time over the graphical representation of the first intraluminal state. In some examples, the display may include a touchscreen.

In additional aspects of the present disclosure, an exemplary medical system comprises a display and includes a user interface that may include graphical elements corresponding to a type of intraluminal state and a first set of medical parameters that may include at least a ureter size, an access sheath size, and a fluid temperature. The medical system may include a processor configured to receive user input selecting a value for the ureter size, a value for the access sheath size, a value for the fluid temperature, and the type of intraluminal state. The type of intraluminal state may be intraluminal pressure, fluid flow rate, and/or body temperature (e.g., renal temperature). The processor may be further configured to determine the intraluminal state over time based on the user input and display on the user interface a graphical representation of the intraluminal state over time. The processor may be further configured to receive user input selecting dynamic visualization of the intraluminal state, and to update the graphical representation based on user input changing the value for the ureter size, the value for the access sheath size, or the value for the fluid temperature.

The present disclosure also includes computer-implemented methods for visualization of an intraluminal state associated with a medical procedure. In some examples, the method includes displaying a user interface that includes graphical elements corresponding to a first set of medical parameters and a type of intraluminal state, receiving, by a processor, a user input selecting a status or value for at least one medical parameter of the first set of medical parameters and selecting the type of intraluminal state, determining, by the processor, the intraluminal state over time based on the user input, and updating, by the processor, the user interface with a graphical representation of the intraluminal state over time.

Any of the systems, devices, and methods disclosed herein may include any of the following features. The type of intraluminal state may be intraluminal pressure, fluid flow rate, or body temperature (e.g., renal temperature). The first set of medical parameters may include at least two medical parameters chosen from a kidney compliance, a ureter size, an access sheath size, a tool designation, a fluid temperature, a laser setting, a fluid bag height, or a cuff pressure. The user interface may include a default value or status for each medical parameter of the first set of medical parameters. The user input may modify the default value of the at least one medical parameter. The intraluminal state may be a first intraluminal state and the user interface may include graphical elements corresponding to a second set of medical parameters that are the same as the medical parameters of the first set of medical parameters. The method may further include receiving, by the processor, user input selecting a status or value for at least one medical parameter of the second set of medical parameters, determining a second intraluminal state over time based on the user input of the status or value for the at least one medical parameter of the second set of medical parameters, and updating the user interface to overlay a graphical representation of the second intraluminal state over time over the graphical representation of the first intraluminal state.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this application, illustrate examples of this disclosure and together with the description, serve to explain the principles of the disclosure.

FIG. 1 depicts an exemplary system, according to aspects of the disclosure.

FIG. 2 depicts a first exemplary user interface for visualization of medical parameters, according to aspects of this disclosure.

FIG. 3 depicts a second exemplary user interface for visualization of medical parameters, according to aspects of this disclosure.

FIG. 4 depicts a method flow diagram, according to aspects of this disclosure.

### DETAILED DESCRIPTION

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values ±10% of a stated value.

Physicians' or other medical professionals' understanding of various parameters that may affect ILP, fluid flow rate during irrigation, and/or body temperature, e.g., renal temperature or the temperature of another organ or other portion of the body, such as bladder temperature, etc., during a medical procedure, e.g., lithotripsy, may help to reduce risk and/or otherwise improve patient outcomes. The systems and methods herein may provide for real-time visualization of various parameters affecting ILP, irrigation flow rate, and/or body temperature (e.g., renal temperature, bladder temperature, etc.).

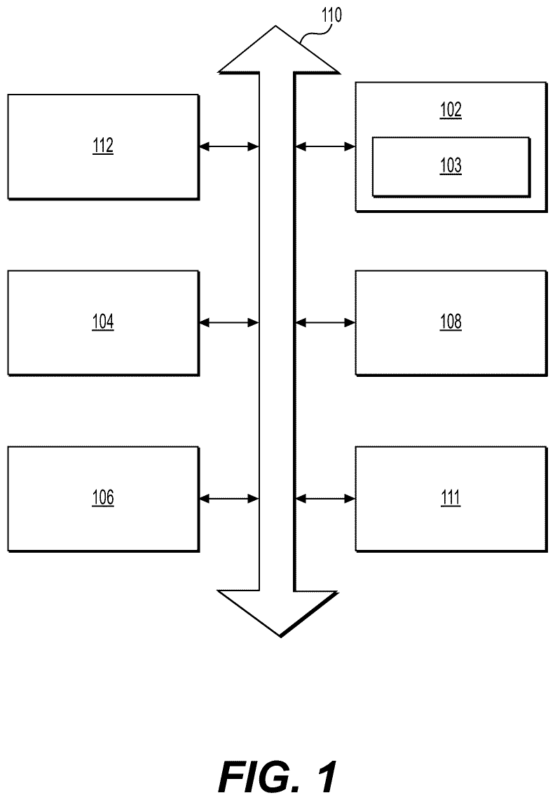

FIG. 1 shows an exemplary system 110 according to aspects of the disclosure. System 110 may include a suitable electronic communications device and/or display device (e.g., a smartphone, a tablet, a personal computer, a smartwatch, a VR/AR headset, a data visualization device, a distributed cloud computing system, etc.). For example, system 110 includes a processor 102, a memory 104, a display 106 (e.g., a display device), an input/output (I/O) device 111, and a transceiver 108. System 110 also may include one or more other components 112 such as, for example, an audio input device, an audio output device, a battery, a data acquisition device, one or more ports to electrically connect system 110 to a power source and/or to other electronic devices, or one or more sensors, etc. The various components of system 110 may be electrically and/or communicatively coupled to one another, e.g., by wired or wireless connections.

Processor 102 may be configured to execute one or more engines for system 110. For example, the engines may include a medical parameter engine 103 for performing operations related to generating a user interface, receiving user input, determining an intraluminal state based on the user input (e.g., applying differential equations relating medical parameters to the intraluminal state), and updating the user interface to display a graphical representation (e.g., a graph or plot) of the intraluminal state over time.

The above referenced engine being an application (e.g., a program) executed by processor 102 is only exemplary. The functionality associated with the engines may also be represented as a separate incorporated component of system 110 or may be a modular component coupled to system 110, e.g., an integrated circuit with or without firmware. For example, the integrated circuit may include input circuitry to receive signals and processing circuitry to process the signals and other information. The engine(s) may also be embodied as one application or separate applications. In addition, in some examples herein, the functionality described for processor 102 is distributed among multiple processors.

Memory 104 may be a hardware component configured to store data related to operations performed by the system 110. Display 106 may be a hardware component configured to show a user interface and optionally receive user input (e.g., via a touchscreen). Optionally, system 110 may include a separate I/O device 111 to allow the user to enter inputs, such as values or status selections for medical parameters associated with a medical procedure. The transceiver 108 may be a hardware component configured to establish a connection with a 5G-NR radio access network (RAN). Accordingly, transceiver 108 may operate on a variety of different frequencies or channels (e.g., sets of consecutive frequencies). Transceiver 108 may send and receive various other types of signaling, such as LTE, satellite, Wi-Fi, Bluetooth, Near Field Communication, and other contemplated future networking standards such as 6G. While transceiver 108 is described as a single component, in some aspects, the transceiver functionality may be distributed among a variety of transceiver modules. Transceiver 108 may include circuitry configured to transmit and/or receive signals (e.g., control signals and data signals). Such signals may be encoded with information implementing any of the systems, devices, and methods described herein. Optionally, processor 102 may be operably coupled to transceiver 108 and configured to receive from and/or transmit signals to transceiver 108. Processor 102 may be configured to encode and/or decode signals for implementing any one of the systems, devices, and methods described herein.

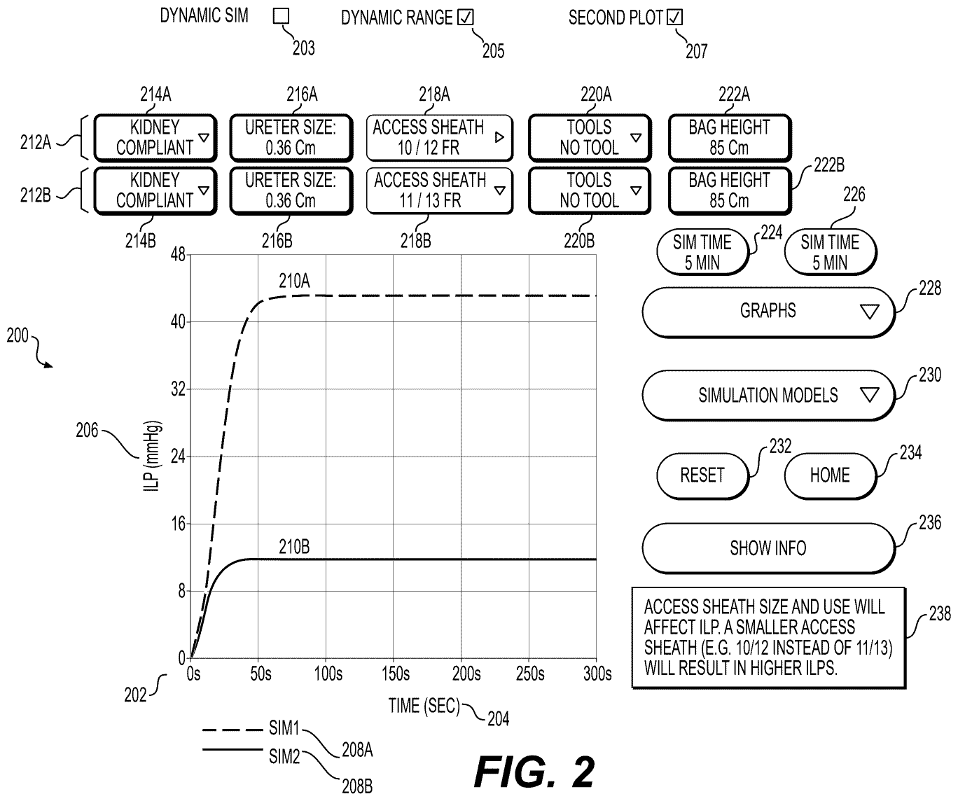

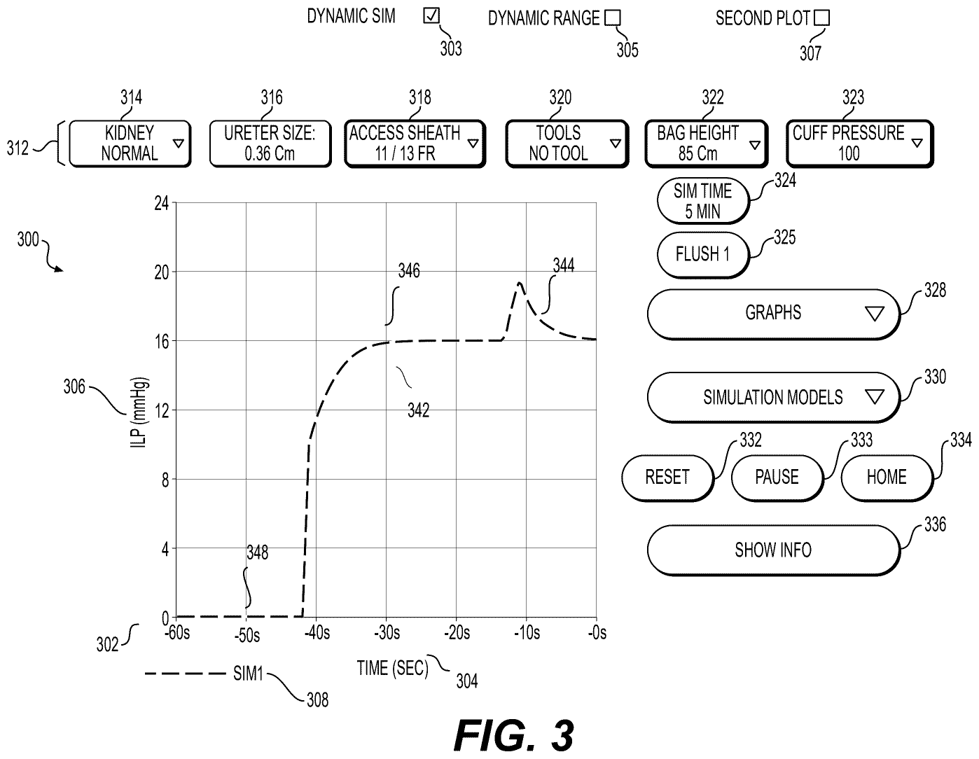

FIG. 2 depicts an exemplary user interface 200 for visualization of parameters associated with a medical procedure, according to aspects of the disclosure. User interface 200 may be displayed by display 106, and may be generated by processor 102. FIG. 2 shows an aspect of the disclosure corresponding to a static visualization of an intraluminal state, such as ILP, fluid flow rate, or body temperature (e.g., renal temperature). For example, FIG. 2 may apply user-selected medical parameter(s) to a mathematical model describing relationships among the medical parameters and intraluminal state. Should the user change the value or status of a parameter, the intraluminal status may be re-determined (recalculated) and the graphical representation updated or replaced with a new graphical representation. A dynamically updating aspect of the disclosure is described with respect to FIG. 3.

As illustrated in FIGS. 2 and 3, the values or status of various parameters may be selectable (or modified from a default value or status) by a user, e.g., using the display or an I/O device in communication with the display. In some aspects of the disclosure, a user may select which parameters are displayed. For example, a user may remove and/or add one or more parameters associated with a medical procedure and/or associated with a timeframe for visualization from the user interface. In some aspects, one or more parameters may be selected, e.g., adjusted from a default value by a user in predefined increments (e.g., 0.02 cm increments for ureter size). In some aspects, the medical parameters may be input by a user precisely (e.g., the user inputting a value for a given parameter). In some aspects, the value(s) of certain parameters may be adjustable in predefined increments relative to an initial or default value, while the value(s) of other parameters may be input by a user. Predefined increments may correspond to dimensions or settings of different medical devices available for performing an intended medical procedure, such as lithotripsy.

Referring again to FIG. 2, user interface 200 may include at least one set of medical parameters, e.g., a first set 212A. Optionally, user interface 200 may include and a second set 212B of medical parameters. The medical parameters in first set 212A and second set 212B may be identical while permitting a user to modify the values or status independently of each other. A user may adjust the value(s) or status of various medical parameters of either first set 212A and/or second set 212B in order to compare the effect modification of the parameter(s) may have on the intraluminal status (e.g., ILP, fluid flow rate, or body temperature such as renal temperature). While two sets of medical parameters are depicted in FIG. 2, the systems, devices, and methods of this disclosure may include a single set of medical parameters or more than two (e.g., three) sets of medical parameters may be available for selection by a user to determine an intraluminal state over time.

Unless explicitly noted, any description of a medical parameter of first set 212A is applicable to corresponding medical parameters of second set 212B. Reference to particular units of measurement throughout the disclosure is exemplary and non-limiting of other suitable units. For example, ureter size is depicted in FIG. 2 (e.g., 216A, 216B) with a unit of centimeters but other units may be used, e.g., millimeters, inches, etc. In some examples, the system 110 may be configured to accept user input to change to one or more other units, e.g., displayed as an initial or default unit for a given parameter.

Exemplary medical parameters that may be included in the systems, devices, and methods include kidney compliance (see discussion below), ureter size, size of access sheath, tool designation (e.g., presence of absence of tools in access sheath and type of tools), height of fluid bag (see discussion below), laser settings (e.g., output power, pulse duration), kidney volume, access sheath working channel cross-sectional shape (e.g., circular, elliptical, etc.), cuff pressure, and/or fluid temperature may also be included as selectable parameters in a user interface and employed in the systems and methods herein.

First set 212A of medical parameters may include a kidney compliance parameter 214A. Kidney compliance may be generally understood as the physical responsiveness of a kidney to fluid filling the collecting system, as different patients' organs may be stiffer or more elastic in response to fluid. A user may assign a kidney compliance score based on the aforementioned responsiveness. For this parameter, the user may select among quantized ranges described by selectable terms such as “compliant”, “normal”, and “rigid”.

First set 212A of medical parameters may include a ureter size parameter 216A. Ureter size may refer to a cross-sectional diameter of the ureter. In some examples, user interface 200 may default to a value between 0.30 cm and 0.40 cm (e.g., typical ureter sizes for many patients), and accept user input to increase or decrease the value by predetermined increments (e.g., ±0.02 cm). For example, the user interface 200 may initially include a ureter size of 0.32 cm, wherein the value may be increased or decreased by the user to select the desired value. Additionally or alternatively, the user may be able to input a value to replace a default value displayed on user interface 200 or to provide a value in absence of a default value.

First set 212A of medical parameters may include an access sheath parameter 218A, corresponding to the size of a working channel of an access sheath used to deliver fluid and/or instruments to a target site. The user may select different sizes of access sheath to explore the effect on intraluminal states in order to assist in planning a medical procedure. The size of access sheath may take into account, for example, patient ureter size, ureter health, etc. Access sheath parameter 218A (e.g., diameter) may be provided in French size, though this is only exemplary. In the exemplary FIG. 2, first access sheath parameter 218A has a smaller value (e.g., 10/12 Fr) compared to second access sheath parameter 218B (e.g., 11/13 Fr). As illustrated by plot 210A corresponding to a higher ILP than plot 210B, a smaller access sheath may result in greater ILP (depending on effects of other medical parameters).

Medical parameter set 212A may include a tool designation parameter 220A. Tool designation parameter 220A may provide a user with the option to select absence of tools (e.g., “no tools”) or to select among a variety of medical tools, such as laser fibers, baskets, balloon dilators, and stents. In some examples, the user interface 200 may include a default of “no tools” for the tool designation parameter 220A. Tool designation parameter 220A may include dimensional/geometric data associated with a selected tool. In general, and depending on the effect of other medical parameters, the presence of tools within the access sheath and/or at the target site may decrease flow rate and ILP.

Medical parameter set 212A may include a fluid bag height parameter 222A. Fluid bag height parameter 222A may correspond to a height at which a fluid container (e.g., a gravity saline bag) is positioned relative to a patient undergoing a medical procedure that includes irrigation via fluid supplied by the container. For example, a target site of the patient may be irrigated by gravity irrigation via a suspended bag. Depending on the effect of other medical parameters, a greater height may correspond to greater ILP and irrigation flow rate due to gravitational potential energy of the fluid.

Additionally or alternatively to the graphical elements shown in FIG. 2, first set 212A may include a fluid temperature (the temperature of a fluid introduced to a patient during a medical procedure, e.g., via irrigation), a laser setting, and/or a cuff pressure. Cuff pressure may refer to a pressure setting used for a pressure cuff applied to an irrigation fluid bag. As with the above medical parameters, each may initially include a default value capable of being modified by a user and/or a user may directly input a selected value. In an example, user interface 200 includes a fluid temperature parameter with a default value of 23° C. that is adjustable by predetermined increments of ±0.2° C. In an example, user interface 200 includes a laser setting parameter that includes a default value for a Holmium fiber laser of 3 J and 40 Hz that is adjustable by predetermined increments of 0.1 J and 10 Hz In an example, user interface 200 includes a cuff pressure parameter that includes a default value of 100 mmHg that is adjustable by predetermined increments of 10 mmHg.

Medical parameters may be employed in a mathematical model to determine the selected intraluminal state, e.g., intraluminal pressure, fluid flow rate, or renal temperature, over time. Exemplary mathematical models are discussed in Williams et al. J. Endourology, vol. 33, pp. 28-34 (2019), Williams et al., World J. Urology, vol. 39, pp. 1707-1716 (2021), and Oratis et al., vol. 13, e0208209 (2018), incorporated by reference herein. The intraluminal state over time may be shown in a graphical representation 202, e.g., a plot or graph. For example, first set 212A of medical parameters may correspond to plot 210A shown in user interface 200, and second set 212B of medical parameters may correspond to plot 210B shown in user interface 200. The differing plots may be depicted with different characteristics (e.g., colors, line styles, etc.) to facilitate distinguishing between data sets. The user may identify plots 210A, 210B by way of reference to key 208A and/or key 208B, respectively.

Graphical representation 202 may include a time interval 204, corresponding to the x-axis. In some examples, time interval 204 may be adjustable by the user, e.g., via simulation time adjustment parameter 224 (corresponding to plot 210A) and/or simulation time parameter 226 (corresponding to plot 210B) in this example.

A dynamic visualization parameter 203 may be selectable by a user change the simulation from a static to dynamic simulation and vice-versa. In the static simulation, parameters (e.g., first set 212A of medical parameters) may be applied to a mathematical model to determine an intraluminal state and display on a graphical representation (e.g., plot 210A). Further description of dynamic simulation is described below with respect to FIG. 3. In the static case depicted in FIG. 2, dynamic visualization parameter 203 is not selected. In some aspects, user interface 200 does not include a dynamic visualization parameter 203.

A dynamic range parameter 205 may allow the user to instruct the system (e.g., processor 102) to automatically adjust the y-axis scale corresponding to a value of the selected intraluminal state to automatically accommodate a plot larger than one or more additional plots on graphical representation 202. In some aspects, user interface 200 does not include dynamic range parameter 205.

A second plot parameter 207 may be selectable by the user to prompt display of a second set of medical parameters (e.g., second set 212B). When second set 212B is shown, the user may select one or more medical parameters of second set 212B in order to determine a second intraluminal state for comparison to the determined first intraluminal state (e.g., comparison of plot 210B against plot 210A). In FIG. 2, second plot parameter 207 is depicted as selected, corresponding to the presence of medical parameter set 212B and plot 210B. In some aspects, user interface 200 does not include second plot parameter 207.

Graphical representation 202 includes a y-axis 206 corresponding to a value of the intraluminal state selected. In some examples, the range of y-axis 206 may be adjustably by input from the user. Graphical representation 202 may display the selected intraluminal state in applicable units, e.g., ILP measured in mmHg, fluid flow rate measured in mL/min or body temperature (e.g., renal temperature or the temperature of another target site within a patient undergoing a medical procedure) measured in ° C.

User interface 200 may provide the option to reset graphical representation 202. For example, the user may reset graphical representation 202 via reset button 232, e.g., to remove plot 210A and plot 210B from the display. Additionally or alternatively, the user may select home button 234 to return to default values for medical parameters and/or other parameters. In some aspects, certain parameters may be adjustable via a menu accessible via home button 243. For example, in some aspects, medical parameters corresponding to patient data, such as ureter size or kidney compliance, may be selectable via the menu accessible via home button 234. Such patient data may be static for a given procedure/simulation.

In some examples, user interface 200 may include a “show info” button 236 configured to toggle (e.g., change from a first configuration to a second configuration) a text box 238. Text box 238 may display relevant information to the user based on a most recently selected medical parameter. For example, if the user most recently adjusted access sheath parameter 218A and/or access sheath parameter 218B, text box 238 may include text to explain the effect the given parameter has on the currently selected dependent variable (e.g., y-axis 206 and graph parameter 228). In some aspects, user interface 200 includes a simulation model 230 option to allow a user to select among different computational models to determine the intraluminal state.

FIG. 3 depicts another exemplary user interface 300 for visualization of medical parameters, showing a dynamically updated graphical representation. For example, a processor of the system (e.g., processor 102 of system 110) may periodically determine a value of the selected intraluminal state (e.g., ILP, fluid flow rate, or body temperature such as renal temperature) over time based on user-selected medical parameters.

User interface 300 includes features similar to those of user interface 200 and may include any of the capabilities discussed above with respect to user interface 200 unless otherwise stated. For example, user interface 300 includes a set 312 of medical parameters that may include a kidney compliance parameter 314, a ureter size parameter 316, an access sheath parameter 318, a tool designation parameter 320, and a fluid bag height parameter 322, which may be substantially similar to respective parameters 212, 214, 216, 218, 220, and 222 described above.

User interface 300 includes a graphical representation 302 with elapsed time 304 on the x-axis and the selected intraluminal state 306 on the y-axis. User interface 300 may be dynamically updated based on user input selecting different values and/or status information for medical parameters of set 312, showing plot 342 with newly calculated values for the selected intraluminal state.

In the example of FIG. 3, the portion leftmost portion of plot 342 (e.g., approximately 40 to 60 seconds previous) may correspond to an initial intraluminal state 348 before the user selects values of status information for medical parameters. A central portion of plot 342 (e.g., approximately 40 to 20 seconds previous) may correspond to the user selecting values or status information for one or more medical parameters of set 312. The values or status information of set 312 may be used in mathematical model to determine the selected intraluminal state (e.g., ILP, fluid flow rate, or body temperature such as renal temperature). In this example, ILP is the selected intraluminal state, shown with a determined value of approximately 17 mmHg. At a time approximately 10 seconds previous in graphical representation graph 302, a peak 344 corresponds to selection of a flush button 325 by the user. Flush button 325 may incorporate the effect of which simulates the impact of a handheld irrigator (e.g. a syringe) on ILP.

The user may update user interface 300 with an additional medical parameter by selecting a second plot parameter 340 as described above in connection to user interface 200. User interface 300 may also be updated with an additional key for the newly generated plot, similar to key 308 corresponding to plot 342. The user may remove the second plot by reselecting second plot parameter 340. Second plot parameter 340 may also be included in a static simulation, such as in FIG. 2.

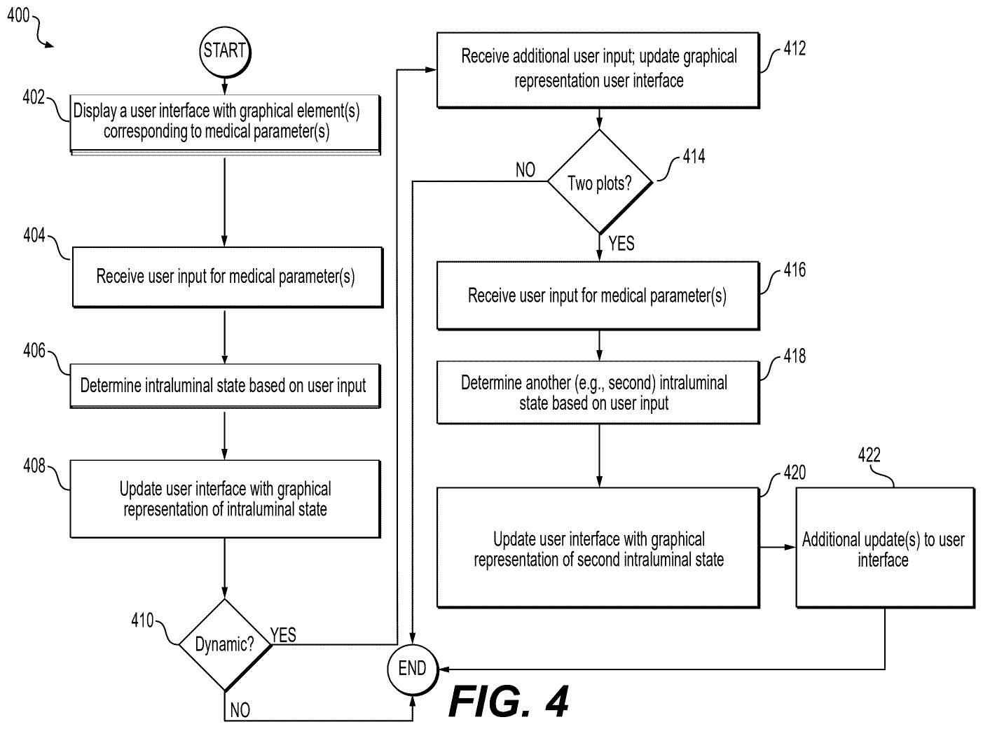

FIG. 4 depicts an exemplary method 400, according to aspects of this disclosure. Method 400 may be performed by system 110 (e.g., one or more steps executed by processor 102 and/or other components of system 110). In step 402, a system (e.g., system 110) may display a user interface (e.g., user interface 200 or 300) on a display with graphical elements corresponding to medical parameters, e.g., a set of medical parameters. In step 404, system 110 may receive user input selecting a value or status for one or more medical parameters and selecting a type of intraluminal state (e.g., ILP, fluid flow rate, or body temperature such as renal temperature). For example, the user may select a value for ureter size and a value for fluid bag height by directly inputting values or by modifying default values initially displayed on the user interface.

In step 406, system 110 may determine an intraluminal state based on the user input. In step 408, system 110 may update the user interface with a graphical representation (e.g., plot or graph) of the determined intraluminal state over time. For example, this may correspond to a plot, such as plot 210A of graphical representation 202 of FIG. 2. In step 410, system 110 may determine whether or not the model is dynamic, e.g., based on user input selecting static visualization or dynamic visualization. System 110 may receive user input to a dynamic visualization parameter, such as dynamic visualization parameter 203. If system 110 receives user input that the simulation is not dynamic (e.g., is static), method 400 may end. If system 110 receives user input that the simulation is dynamic, method 400 continues in step 412.

In step 412, system 110 may perform additional updates to the user interface, e.g., the graphical representation of the intraluminal state over time displayed in the user interface. For example, system 110 may periodically re-determine (e.g., re-calculate) the intraluminal state (e.g., ILP) based on user input for medical parameters. This may be exemplified by the various calculated ILPs over time shown in plot 342 of FIG. 3.

In step 414, system 110 may determine whether an additional intraluminal state is to be determined, e.g., and another plot displayed in the user interface. For example, system 110 may receive user input to second plot parameter 307. If a second plots is not selected, method 400 may end. If the user input selects a second plot, method 400 continues in step 416. It will be understood that if second plot parameter 307 is selected by the user, a second set of medical parameters may be displayed in the user interface for selection by the user. In step 416, system 110 may receive user input of one of more medical parameters in a second set of medical parameters. In step 418, system 110 may determine a second intraluminal state based on the user input for the second set of medical parameters. In step 420, system 110 may update the user interface with a graphical representation of the second intraluminal state over time. In step 422, system 110 may perform additional updates to the user interface based on user input. For example, system 110 may periodically re-determine (e.g., recalculate) the first and/or second intraluminal state(s) based on user input for both the first medical parameter set and the second medical parameter set.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, e.g., may enable loading of the software from one computer or processor into another. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A medical system comprising:
a display including a user interface that includes graphical elements corresponding to a first set of medical parameters and a type of intraluminal state; and
a processor configured to:
receive user input selecting a status or value for at least one medical parameter of the first set of medical parameters and the type of intraluminal state;
determine the intraluminal state over time based on the user input; and
display on the user interface a graphical representation of the intraluminal state over time.

2. The system of claim 1, wherein the type of intraluminal state is intraluminal pressure, fluid flow rate, or body temperature.

3. The system of claim 1, wherein the first set of medical parameters includes at least two medical parameters chosen from a kidney compliance, a ureter size, an access sheath size, a tool designation, a fluid temperature, a laser setting, a fluid bag height, or a cuff pressure.

4. The system of claim 1, wherein the first set of medical parameters includes a ureter size, an access sheath size, a fluid temperature, and a laser setting; and wherein the type of intraluminal state is intraluminal pressure or renal temperature.

5. The system of claim 1, wherein the user interface includes a default value or status for each medical parameter of the first set of medical parameters, the default value or status capable of being modified by a user.

6. The system of claim 5, further comprising a memory that stores the default value or status for each medical parameter.

7. The system of claim 1, wherein determining the intraluminal state over time includes numerically solving differential equations based on the user input selecting the status or value for the at least one medical parameter.

8. The system of claim 1, wherein the processor is further configured to receive user input selecting static visualization or dynamic visualization of the intraluminal state.

9. The system of claim 8, wherein the processor is further configured to receive user input selecting dynamic visualization, and to update the graphical representation based on user input changing a value or status of at least one medical parameter.

10. The system of claim 9, wherein the user interface further includes a graphical element corresponding to a fluid flush, and wherein the user input includes a selection of the fluid flush.

11. The system of claim 1, wherein the user interface includes graphical elements corresponding to a second set of medical parameters that are the same as the medical parameters of the first set of medical parameters.

12. The system of claim 11, wherein the intraluminal state is a first intraluminal state and the processor is further configured to:
receive user input selecting a status or value for at least one medical parameter of the second set of medical parameters;
determine a second intraluminal state over time based on the user input of the status or value for the at least one medical parameter of the second set of medical parameters; and
update the user interface to overlay a graphical representation of the second intraluminal state over time over the graphical representation of the first intraluminal state.

13. The system of claim 1, wherein the display includes a touchscreen.

14. A medical system comprising:
a display including a user interface that includes graphical elements corresponding to a type of intraluminal state and a first set of medical parameters including at least a ureter size, an access sheath size, and a fluid temperature; and
a processor configured to:
receive user input selecting a value for the ureter size, a value for the access sheath size, a value for the fluid temperature, and the type of intraluminal state, wherein the type of intraluminal state is intraluminal pressure, fluid flow rate, or body temperature;
determine the intraluminal state over time based on the user input; and
display on the user interface a graphical representation of the intraluminal state over time.

15. The system of claim 14, wherein the processor is further configured to receive user input selecting dynamic visualization of the intraluminal state, and to update the graphical representation based on user input changing the value for the ureter size, the value for the access sheath size, or the value for the fluid temperature.

16. A computer-implemented method for visualization of an intraluminal state associated with a medical procedure, the method comprising:
displaying a user interface that includes graphical elements corresponding to a first set of medical parameters and a type of intraluminal state;
receiving, by a processor, a user input selecting a status or value for at least one medical parameter of the first set of medical parameters and selecting the type of intraluminal state;
determining, by the processor, the intraluminal state over time based on the user input; and
updating, by the processor, the user interface with a graphical representation of the intraluminal state over time.

17. The method of claim 16, wherein the type of intraluminal state is intraluminal pressure, fluid flow rate, or renal temperature.

18. The method of claim 16, wherein the first set of medical parameters includes at least two medical parameters chosen from a kidney compliance, a ureter size, an access sheath size, a tool designation, a fluid temperature, a laser setting, a fluid bag height, or a cuff pressure.

19. The method of claim 16, wherein the user interface includes a default value or status for each medical parameter of the first set of medical parameters, and wherein the user input modifies the default value of the at least one medical parameter.

20. The method of claim 16, wherein the intraluminal state is a first intraluminal state, and the user interface includes graphical elements corresponding to a second set of medical parameters that are the same as the medical parameters of the first set of medical parameters, the method further comprising:
receiving, by the processor, user input selecting a status or value for at least one medical parameter of the second set of medical parameters;
determining a second intraluminal state over time based on the user input of the status or value for the at least one medical parameter of the second set of medical parameters; and
updating the user interface to overlay a graphical representation of the second intraluminal state over time over the graphical representation of the first intraluminal state.
